(19) United States
(12) Patent Application Publication
(10) Pub. No.: US 2026/0146030 A1
(43) Pub. Date: May 28, 2026

(54) POST-TREATMENT METHOD FOR SAFLUFENACIL-CONTAINING REACTION SOLUTION

(71) Applicant: SHANDONG WEIFANG RAINBOW CHEMICAL CO.,LTD, Shandong (CN)

(72) Inventors: Zhiqing LI, Shandong (CN); Xinghui WEI, Shandong (CN); Ziyong LI, Shandong (CN); Zhongjie LIU, Shandong (CN); Luke MA, Shandong (CN)

(73) Assignee: SHANDONG WEIFANG RAINBOW CHEMICAL CO.,LTD, Shandong (CN)

(21) Appl. No.: 19/454,266

(22) Filed: Jan. 20, 2026

Related U.S. Application Data
(63) Continuation of application No. PCT/CN2025/133238, filed on Nov. 7, 2025.

(30) Foreign Application Priority Data
Nov. 15, 2024 (CN) 202411638658.5

(51) Int. Cl. C07D 239/54

(52) U.S. Cl. CPC C07D 239/54

(57) ABSTRACT

The present application relates to the field of pesticide chemical industry technology, and discloses a post-treatment method for a saflufenacil-containing reaction solution. The post-treatment method includes the following steps: (1) after phase separation of a saflufenacil-containing reaction solution, performing desolvation treatment on an organic phase, and then adding a first organic solvent for stirring; (2) after the stirring is completed, performing filtration, leaching an obtained filter cake with a second organic solvent, and obtaining saflufenacil after drying. The post-treatment method provided by the present application can simply and efficiently treat the saflufenacil-containing reaction solution, with a product yield of over 90% and a content of about 98%.

### CROSS-REFERENCE TO RELATED APPLICATION

This application is a continuation of international application of PCT application serial no. PCT/CN2025/133238 filed on Nov. 7, 2025, which claims the priority benefit of China application no. 202411638658.5 filed on Nov. 15, 2024. The entirety of each of the above mentioned patent applications is hereby incorporated by reference herein and made a part of this specification.

### BACKGROUND

### Technical Field

The present application relates to the field of pesticide chemical industry technology, and specifically relates to a post-treatment method for a saflufenacil-containing reaction solution.

### Description of Related Art

Saflufenacil belongs to the pyrimidine class, is a protoporphyrinogen oxidase (PPO) inhibitor, can be used as a non-selective herbicide, and can effectively control a variety of broadleaf weeds, including weeds resistant to glyphosate, ALS, and triazine. Saflufenacil has a rapid non-selective effect and rapid degradation of soil residues; it can be mixed with gramineous weed herbicides (e.g., glyphosate), with good herbicidal effect, and can be applied in various crop fields and non-cultivated lands, with small rotation restrictions.

The existing production process of saflufenacil results in large product loss, and the yield is only 84% after a series of post-treatment. With the continuous decline of the pesticide market, there are increasingly higher requirements for the yield of technical material production.

Chinese patent CN 101821233A discloses a method for producing saflufenacil. The post-treatment process disclosed in this patent is: after completion of the reaction, the phases are separated, the organic phase is dried and part of the solvent is removed; during cooling, saflufenacil crystallizes out, which is filtered, washed with toluene, and dried. This method uses a large amount of organic solvent toluene for crystallization and leaching, resulting in a large content of technical material in the toluene mother liquor, difficult to recover, with a yield of only 84%.

A synthesis method of saflufenacil is reported in an international journal J Heterocyclic Chem. 2020, 57, 151-156, and the post-treatment method of this report is as follows: the mixture is allowed to stand and separate; the toluene layer is collected, the mixture is washed with water (100 mL×2), and dried over anhydrous Na₂SO₄; most of the solvent is removed, heptane (150 mL) is added and stirred at room temperature for 2 h, the resulting solid is collected by filtration, the mixture is washed with heptane (100 mL×1), and dried under vacuum at 40° C. to obtain saflufenacil with a yield of 86.1%.

BASF discloses a method for recovering technical material from saflufenacil mother liquor in international patent WO 2023/232507A1. The patent describes that the proportion of technical material in the mother liquor is about 10%, and the recovery process is complicated: it is first necessary to add water to the mother liquor, then adjust pH>8 to extract the target product into the aqueous phase, then add an organic solvent to the aqueous phase, then adjust pH<2 to back-extract saflufenacil into the organic phase. The product obtained in this way, however, contains a large number of impurities, which require secondary crystallization. In addition, too strong alkalinity in the extraction process easily leads to the decomposition of saflufenacil, and its decomposition equation is as follows:

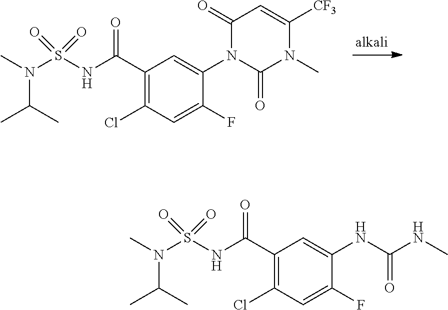

Therefore, it is urgent to develop a post-treatment method for a saflufenacil-containing reaction solution with simple operation and improved yield.

### SUMMARY

The object of the present application is to overcome the problems existing in the prior art and provide a post-treatment method for a saflufenacil-containing reaction solution.

In order to achieve the above object, the present application provides a post-treatment method for a saflufenacil-containing reaction solution, wherein the post-treatment method includes the following steps:
(1) after phase separation of a saflufenacil-containing reaction solution, performing desolvation treatment on an organic phase, and then adding a first organic solvent for stirring; and
(2) after the stirring is completed, performing filtration, leaching an obtained filter cake with a second organic solvent, and obtaining pure saflufenacil after drying.

With the above technical solution, the beneficial technical effects achieved by the present application are as follows:
(1) the post-treatment method provided by the present application can simply and efficiently treat the saflufenacil-containing reaction solution, with a product yield of over 90% and a content of about 98%;
(2) after post-treatment by the method of the present application, the loss of saflufenacil technical material is less than 1%;
(3) the organic solvents used in the post-treatment of this method can be recovered by simple distillation, with a recovery rate of over 98%, and can be reused.

### BRIEF DESCRIPTION OF THE DRAWINGS

FIG. 1 is an HPLC chromatogram of saflufenacil obtained in Example 1 of the present application.

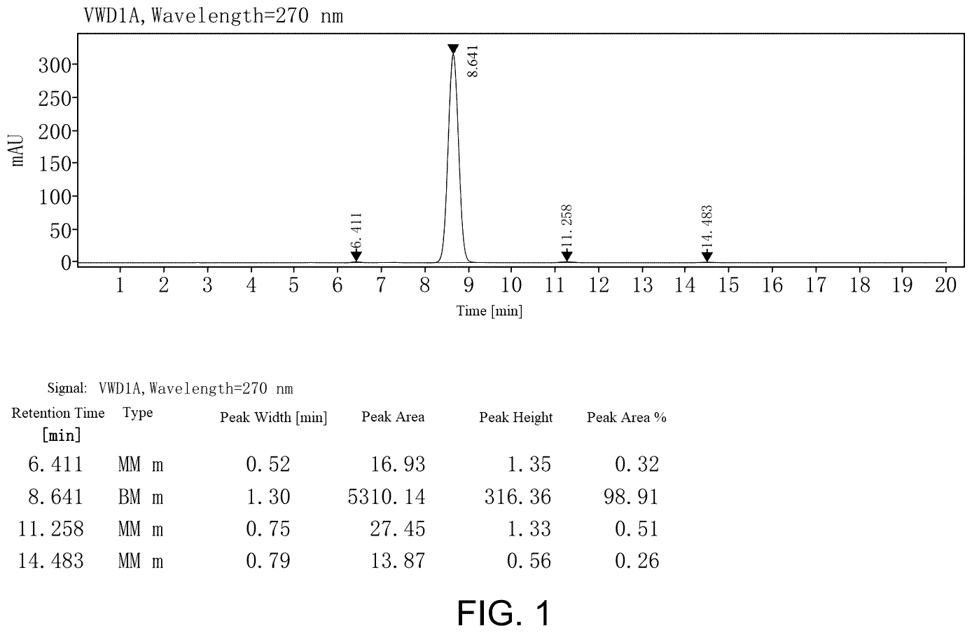

FIG. 2 is an HPLC chromatogram of the isooctanol mother liquor obtained in Example 1 of the present application.

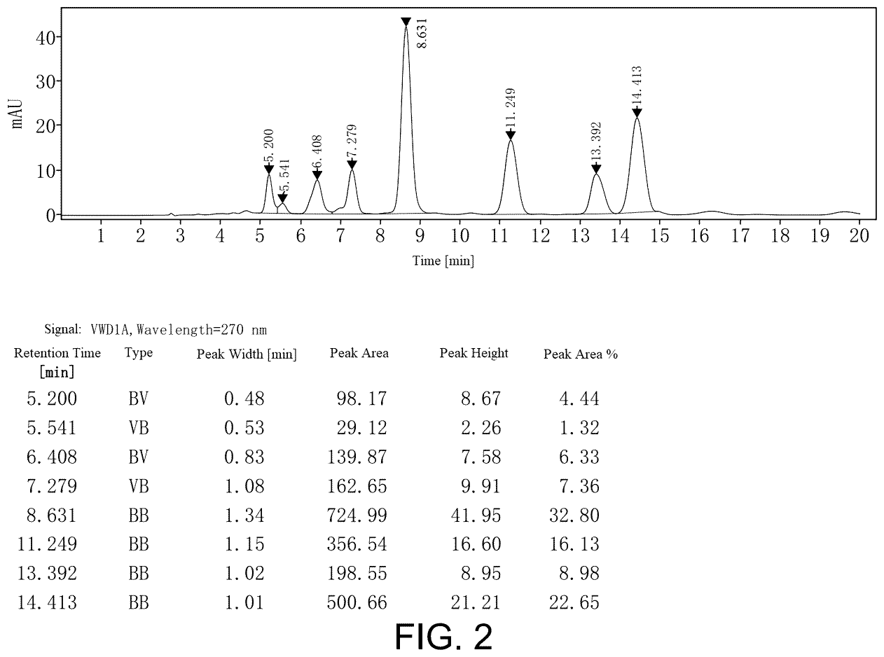

### DESCRIPTION OF THE EMBODIMENTS

The endpoints of the ranges and any values disclosed herein are not limited to the precise range or value, and these ranges or values should be understood to encompass values approximating these ranges or values. For numerical ranges, between the endpoints of each range, between the endpoints of each range and individual point values, and between individual point values can be combined with each other to produce one or more new numerical ranges, and such numerical ranges are considered to be specifically disclosed herein.

The yields described in the present application are molar yields.

According to a first aspect of the present application, there is provided a post-treatment method for a saflufenacil-containing reaction solution, wherein the post-treatment method includes the steps of:
(1) after phase separation of a saflufenacil-containing reaction solution, performing desolvation treatment on an organic phase, and then adding a first organic solvent for stirring;
(2) after the stirring is completed, performing filtration, leaching an obtained filter cake with a second organic solvent, and obtaining saflufenacil after drying.

For the saflufenacil-containing reaction solution, the traditional post-treatment method uses toluene for stirring, resulting in a high content of the technical material in the mother liquor, low yield of the technical material, complex operation, and large losses. In the present application, by mixing and stirring the desolvated crude saflufenacil with a first organic solvent (e.g., isooctanol), the problem of large post-treatment loss during the synthesis of the technical material is solved, so that the yield of the product saflufenacil is increased from 84% to more than 90%, the content is about 98%, and the loss ratio of the technical saflufenacil is 0.7-1.0% (relative to the theoretical mass of saflufenacil).

In some embodiments of the present application, the saflufenacil-containing reaction solution is prepared by the following method:
adding 2-chloro-5-[3,6-dihydro-2,6-dioxo-4-(trifluoromethyl)-1(2H)-pyrimidinyl]-4-fluoro-N-{[methyl-(1-methylethyl)amino]sulfonyl}benzamide, a phase transfer catalyst, and a methylating agent into a reaction solvent, and controlling a reaction pH to 4-6 with liquid caustic soda to perform a reaction to obtain the saflufenacil-containing reaction solution.

In some embodiments of the present application, the phase transfer catalyst is tetrabutylammonium bromide.

In some embodiments of the present application, the methylating agent is dimethyl sulfate.

For the preparation of the saflufenacil-containing reaction solution, reference can be made to CN 101821233A, for example, the scheme described in Example 2 thereof.

The reaction scheme is as follows:

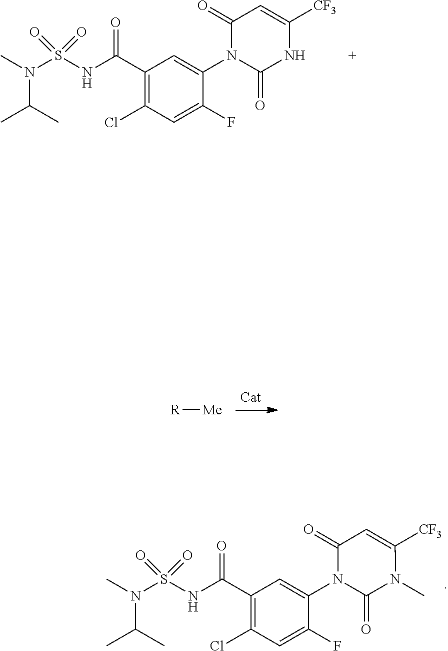

In some embodiments of the present application, the first organic solvent and the second organic solvent are each independently selected from alcohols, esters, or ethers with not less than 7 carbon atoms, preferably alcohols, esters, or ethers with not less than 8 carbon atoms.

In some embodiments of the present application, the first organic solvent and the second organic solvent are each independently at least one selected from the group consisting of 1-heptanol, anisole, n-octanol, isooctanol, 2-octanol, hexyl acetate, and 1-nonanol, preferably isooctanol and/or hexyl acetate.

In some embodiments of the present application, the first organic solvent and the second organic solvent are of the same type.

In some embodiments of the present application, hydrochloric acid is used for acidification until a pH of an aqueous layer of the reaction solution is 1-2, and negative pressure desolvation treatment is performed on the organic phase at 40-45° C. The purpose of adjusting the pH of the aqueous layer is to stabilize saflufenacil.

In some embodiments of the present application, a condition for the negative pressure is from −0.09 MPa to −0.1 MPa.

In some embodiments of the present application, a ratio of the theoretical mass of saflufenacil to the mass of the first organic solvent is 1:1-10, such as 1:1, 1:2, 1:3, 1:4, 1:5, 1:6, 1:7, 1:8, 1:9, 1:10, and any value within an range consisting of any two of the foregoing values, preferably 1:1-6, further preferably 1:2.

In some embodiments of the present application, a temperature for the stirring in step (1) is 0-80° C., preferably 40-45° C.

In some embodiments of the present application, a stirring time in step (1) is 20-40 min, preferably 30 min.

In some embodiments of the present application, a ratio of the theoretical mass of saflufenacil to the mass of the second organic solvent is 1:0.5-2, preferably 1:1.

In some embodiments of the present application, a temperature for the drying in step (2) is 50-90° C., preferably 75° C.

In some embodiments of the present application, a drying time in step (2) is 4-8 h, preferably 5 h.

In some embodiments of the present application, in step (2), after the stirring is completed, a temperature for the system is lowered to 15-20° C., and then the filtration is performed.

According to a particularly preferred embodiment of the present application, a post-treatment method for saflufenacil-containing reaction solution includes the following steps:
the saflufenacil-containing reaction solution is acidified with hydrochloric acid until the pH of the aqueous layer of the reaction solution reaches 1-2, and the organic phase is subjected to negative pressure (P=from −0.09 MPa to −0.1 MPa) desolvation treatment at 40-45° C.; after the organic solvent in the reaction solution is completely removed, 2 times the mass of a first organic solvent (based on the theoretical mass of saflufenacil) is added; after the addition of the first organic solvent, stirring treatment is performed at 40-45° C. for 30 min; the system temperature is lowered to 15-20° C. and then suction filtration is performed, and the filter cake is leached with a second organic solvent (0.5 times mass*2, based on the theoretical mass of saflufenacil); the wet saflufenacil product is dried at 75° C. for 5 h to obtain saflufenacil; the mother liquor (filtrate+leachate) is directly subjected to distillation recovery, and the recovered organic solvent can be directly used for recycling stirring.

Hereinafter, the present application will be described in detail by way of examples.

For the following examples and comparative examples where specific conditions are not specified, they are conducted according to conventional conditions or conditions suggested by the manufacturer. The reagents or instruments used, for which the manufacturer is not specified, are conventional products that can be obtained through commercial sources.

### Example 1

First, 40.0 g (0.0785 mol, content 95.5%) of 2-chloro-5-[3,6-dihydro-2,6-dioxo-4-(trifluoromethyl)-1(2H)-pyrimidinyl]-4-fluoro-N-{[methyl-(1-methylethyl)amino]sulfonyl}benzamide, 2.5 g (0.0078 mol) of tetrabutylammonium bromide (=TBAB) and 13.4 g (0.106 mol) of dimethyl sulfate were added to a mixture of toluene, water and THF (tetrahydrofuran) at 25° C., and the mixture was heated to 40° C. Then, a pH of 5.3-5.5 was achieved in the reaction mixture by adding 10% aqueous NaOH solution. The mixture was stirred at 40° C. for 1 h, during which the addition of 10% aqueous NaOH solution was continued to keep the pH constant at 5.3-5.5. The addition of 10% aqueous NaOH solution was stopped after 1 h, at which point the pH dropped back to 4.4-4.5. The mixture was further stirred at a pH of 4.4-4.5 and 40° C. for 5.5 h to obtain a saflufenacil-containing reaction solution.

The saflufenacil-containing reaction solution was acidified with hydrochloric acid until the pH of the aqueous layer of the reaction solution reached 1, the aqueous layer was washed with toluene, the organic phases were combined, and the organic phase was subjected to negative pressure (P=−0.1 MPa) desolvation treatment at 45° C.; after the organic solvent in the reaction solution was completely removed, 78.6 g of isooctanol was added, and after the addition of isooctanol, stirring treatment was performed at 45° C. for 30 min; the system temperature was lowered to 20° C., followed by suction filtration, and the filter cake was leached with isooctanol (19.6 g*2); the obtained wet saflufenacil product was dried at 75° C. for 5 h to obtain 38.1 g of saflufenacil with a content of 98.2% and a yield of 95.1%. The filtrate obtained by suction filtration and the leachate after leaching were combined into an isooctanol mother liquor, in which the ratio of saflufenacil technical material was 0.7% (relative to the theoretical mass of saflufenacil).

### Example 2

First, 60 g (0.1177 mol, content 95.5%) of 2-chloro-5-[3,6-dihydro-2,6-dioxo-4-(trifluoromethyl)-1(2H)-pyrimidinyl]-4-fluoro-N-{[methyl-(1-methylethyl)amino]sulfonyl}benzamide, 3.9 g (0.0118 mol) of tetrabutylammonium bromide (=TBAB) and 20.2 g (0.159 mol) of dimethyl sulfate were added to a mixture of toluene, water and THF (tetrahydrofuran) at 25° C., and the mixture was heated to 40° C. Then, a pH of 5.3-5.5 was achieved in the reaction mixture by adding 10% aqueous NaOH solution. The mixture was stirred at 40° C. for 1 h, during which the addition of 10% aqueous NaOH solution was continued to keep the pH constant at 5.3-5.5. The addition of 10% aqueous NaOH solution was stopped after 1 h, at which point the pH dropped back to 4.4-4.5. The mixture was further stirred at a pH of 4.4-4.5 and 40° C. for 5.5 h to obtain a saflufenacil-containing reaction solution.

The saflufenacil-containing reaction solution was acidified with hydrochloric acid until the pH of the aqueous layer reached 1.5, the aqueous layer was washed with toluene, the organic phases were combined, and the organic phase was subjected to negative pressure (P=−0.1 MPa) desolvation treatment at 40° C.; after the organic solvent in the reaction solution was completely removed, 117.9 g of isooctanol was added; after the addition of isooctanol, the mixture was stirred at 40° C. for 30 min; the system temperature was lowered to 20° C., followed by suction filtration, and the filter cake was leached with isooctanol (29.5 g*2); the obtained wet saflufenacil product was dried at 75° C. for 5 h to obtain 56.8 g of saflufenacil with a content of 98.5% and a yield of 95.0%. The filtrate obtained by suction filtration and the leachate after leaching were combined into an isooctanol mother liquor, in which the ratio of saflufenacil technical material was 0.9% (relative to the theoretical mass of saflufenacil).

### Example 3

The method of Example 1 was followed to obtain saflufenacil, except that isooctanol was replaced with hexyl acetate.

Saflufenacil 38.0 g, with a content of 98.2% and a yield of 95.0%, was obtained. The proportion of saflufenacil technical material in the obtained hexyl acetate mother liquor was 1.0% (relative to the theoretical mass of saflufenacil).

### Example 4-9

The method of Example 1 was followed to obtain saflufenacil, except that isooctanol was replaced with other organic solvents. The yields are shown in Table 1.

TABLE 1

| No. | Solvent classification | Solvent type | Yield/% |
|---|---|---|---|
| Example 4-1 | 4 carbons and below | Methanol | 67.3 |
| Example 4-2 | | Ethanol | 70.6 |
| Example 4-3 | | Propanol | 69.7 |
| Example 4-4 | | Butanol | 74.4 |
| Example 4-5 | | Isopropanol | 72.9 |
| Example 5-1 | 5 carbons | Propyl acetate | 77.1 |
| Example 5-2 | | Isopropyl acetate | 73.5 |
| Example 5-3 | | Methyl tert-butyl ether | 76.5 |
| Example 6-1 | 6 carbons | 1-Hexanol | 83.0 |
| Example 6-2 | | Butyl acetate | 88.4 |
| Example 6-3 | | Isopropyl ether | 81.0 |
| Example 7-1 | 7 carbons | 1-Heptanol | 92.7 |
| Example 7-2 | | Anisole | 88.3 |
| Example 8-1 | 8 carbons | n-Octanol | 94.9 |
| Example 8-2 | | 2-Octanol | 95.2 |
| Example 9 | 9 carbons | 1-Nonanol | 95.1 |

### Examples 10-13

The method of Example 1 was followed to obtain saflufenacil, except that the mass of isooctanol used during stirring was replaced with other masses, respectively. The yield and content of saflufenacil are shown in Table 2.

TABLE 2

| | | Theoretical mass of | | |
|---|---|---|---|---|
| | Isooctanol | saflufenacil:isooctanol | | |
| No. | mass/g | mass | Yield/% | Content/% |
| Example 10 | 39.3 | 1:1 | 95.5 | 97.3 |
| Example 11 | 157.2 | 1:4 | 94.9 | 98.0 |
| Example 12 | 235.8 | 1:6 | 94.3 | 98.2 |
| Example 13 | 393.0 | 1:10 | 93.6 | 98.5 |

The preferred embodiments of the present application have been described in detail above; however, the present application is not limited thereto. Within the scope of the technical concept of the present application, various simple modifications can be made to the technical solution of the present application, including combining the respective technical features in any other suitable manner; these simple modifications and combinations shall also be regarded as the content disclosed by the present application and all fall within the protection scope of the present application.

### INDUSTRIAL APPLICABILITY

The present application discloses a post-treatment method for a saflufenacil-containing reaction solution. The post-treatment method includes the following steps: (1) after phase separation of a saflufenacil-containing reaction solution, performing desolvation treatment on an organic phase, and then adding a first organic solvent for stirring; (2) after the stirring is completed, performing filtration, leaching an obtained filter cake with a second organic solvent, and obtaining saflufenacil after drying. The post-treatment method provided by the present application can simply and efficiently treat the saflufenacil-containing reaction solution, with a product yield of over 90% and a content of about 98%.

What is claimed is:

1. A post-treatment method for a saflufenacil-containing reaction solution, wherein the post-treatment method comprises the following steps:
(1) after phase separation of a saflufenacil-containing reaction solution, performing desolvation treatment on an organic phase, and then adding a first organic solvent for stirring; and
(2) after the stirring is completed, performing filtration, leaching an obtained filter cake with a second organic solvent, and obtaining saflufenacil after drying.

2. The post-treatment method according to claim 1, wherein the saflufenacil-containing reaction solution is prepared by the following method:
adding 2-chloro-5-[3,6-dihydro-2,6-dioxo-4-(trifluoromethyl)-1(2H)-pyrimidinyl]-4-fluoro-N-{[methyl-(1-methylethyl)amino]sulfonyl}benzamide, a phase transfer catalyst and a methylating agent into a reaction solvent, and controlling a reaction pH to 4-6 with liquid caustic soda to perform a reaction to obtain the saflufenacil-containing reaction solution.

3. The post-treatment method according to claim 2, wherein the phase transfer catalyst is tetrabutylammonium bromide;
preferably, the methylating agent is dimethyl sulfate.

4. The post-treatment method according to claim 1, wherein the first organic solvent and the second organic solvent are each independently selected from alcohols, esters, or ethers with not less than 7 carbon atoms, preferably alcohols, esters, or ethers with not less than 8 carbon atoms;
preferably, the first organic solvent and the second organic solvent are each independently at least one selected from the group consisting of 1-heptanol, anisole, n-octanol, isooctanol, 2-octanol, hexyl acetate, and 1-nonanol, preferably isooctanol and/or hexyl acetate;
preferably, the first organic solvent and the second organic solvent are of the same type.

5. The post-treatment method according to claim 1, wherein hydrochloric acid is used for acidification until a pH of an aqueous layer of the reaction solution is 1-2, and negative pressure desolvation treatment is performed on the organic phase at 40-45° C.;
preferably, a condition for the negative pressure is from −0.09 MPa to −0.1 MPa.

6. The post-treatment method according to claim 1, wherein a ratio of the theoretical mass of saflufenacil to the mass of the first organic solvent is 1:1-10, preferably 1:1-6, and more preferably 1:2.

7. The post-treatment method according to claim 1, wherein a stirring temperature in step (1) is 0-80° C., preferably 40-45° C.;
preferably, a stirring time in step (1) is 20-40 min, preferably 30 min.

8. The post-treatment method according to claim 1, wherein a ratio of the theoretical mass of saflufenacil to the mass of the second organic solvent is 1:0.5-2, preferably 1:1.

9. The post-treatment method according to claim 1, wherein a drying temperature in step (2) is 50-90° C., preferably 75° C.;
preferably, a drying time in step (2) is 4-8 h, preferably 5 h.

10. The post-treatment method according to claim 1 wherein in step (2), after the stirring is completed, a system temperature is lowered to 15-20° C. and then the filtration is performed.
